(19) United States
(12) Patent Application Publication
(10) Pub. No.: US 2023/0218308 A1
(43) Pub. Date: Jul. 13, 2023

(54) Patient-specific Hemivertebral Surgical Guide

(71) Applicant: S.M.A.I.O., Saint-Priest (FR)

(72) Inventors: Jean-Charles Roussouly, Poleymieux (FR); Jérémy Gallien, Lyon (FR)

(73) Assignee: S.M.A.I.O., Saint-Priest (FR)

(21) Appl. No.: 18/152,757

(22) Filed: Jan. 11, 2023

(30) Foreign Application Priority Data
Jan. 11, 2022 (FR) 2200176

(51) Int. Cl. A61B 17/17

(52) U.S. Cl. CPC A61B 17/1757; A61B 2017/568

(57) ABSTRACT

The patient-specific hemivertebral surgical guide has a body with an aiming passage for guiding an instrument and a coupling face, arranged on a distal side of the body and specific to a patient so as to be applied in bespoke contact with the bone material of only one half, either left or right, of the vertebra. The coupling face has a morphology-adapted surface congruent with the posterior face of the lamina of the half of the vertebra and including peripheral portions located respectively in anteroposterior line with the lower edge of the lamina and the lateral edge of the isthmus of the half of the vertebra, and positioning studs protruding correspondingly from the peripheral portions. The morphology-adapted surface and the positioning studs are shaped so as to cooperate by matching shapes with the posterior face of the lamina, the lower edge of the lamina and the lateral edge of the isthmus, respectively, so as to maintain the body in a unique position.

### FIELD

The present invention provides a patient-specific hemivertebral surgical guide.

### BACKGROUND

In spinal surgery, a major challenge for a successful surgery is the precise placing of implants, such as screws, through spinal bone structures along trajectories, each associating an entry point and a direction, which are difficult to determine with the bare eye. Indeed, each of the vertebrae forming the spine of a given patient has a specific geometry unique to each patient. Although surgeons are trained to perform the corresponding surgical procedures, the unique geometry of each vertebra and the limited space for inserting bone implants bear a persistent unpredictable risk, potentially involving the occurrence of neurological and/or hemorrhagic complications. Surgeons commonly use different techniques for securing the correct positioning of implants.

One of such techniques consists in using surgical guides that are specific to the patient being operated on, during the intraoperative time. Such a surgical guide is provided with morphology-adapted elements, specific to the patient operated on, which are designed for being positioned, during the intraoperative time, congruently on anatomical landmarks of at least one of the vertebrae of the patient, associated with the surgical guide. Such morphology-adapted elements of the surgical guide are defined and manufactured preoperatively using mapping data relating to the vertebra associated with the surgical guide, coming e.g. from CT images of the patient's spine.

In practice, the surgical guides available on the market have the shape of an arch, which spans the spinous process of the vertebra and the ends of which incorporate the aforementioned morphology-adapted elements so that the latter are applied to areas of the vertebra, located on either side of the spinous process of the vertebra. The arch carries, on each of the lateral sides thereof, an aiming passage for guiding a surgical instrument handled by the surgeon, such as a bone drilling tool. Each of the aiming passages is arranged on the arch in such a way that, when the surgical guide is applied to the vertebra, the surgical instrument is guided with respect to the vertebra through the aiming passage along a predetermined trajectory that has been planned preoperatively using the aforementioned mapping data.

The use of such patient-specific surgical guides provides high aiming precision, without the need to irradiate the patient during surgery. Nevertheless, such surgical guides have technical limitations. Indeed, such surgical guides require positioning on anatomical landmarks distributed on both sided of the spinous process of the vertebra and thus widely cover the posterior side of the vertebra. As a result, the surgeon's vision towards the surgical site is greatly obstructed. Moreover, to use such surgical guides, surgery has to be performed with a bilateral open approach, which prohibits the use thereof in minimally invasive surgical procedures wherein the surgeon makes only a small incision for positioning the implants.

### SUMMARY

The purpose of the present invention is to propose a novel surgical guide which, while providing precise aiming, is both practical to use and compact for limiting the surgical approach.

To this end, the subject matter of the invention is a patient-specific hemivertebral surgical guide. The surgical guide includes a body which is provided (i) with an aiming passage, which links proximal and distal sides of the body to each other and which is suitable for guiding a surgical instrument, such as a bone drilling tool or a bone implant tool, and (ii) with a coupling face, which is arranged on the distal side of the body and is patient-specific to be applied in bespoke contact with bone material of only one half, either left or right, of a vertebra of the patient. The coupling face has a morphology-adapted surface, which is congruent with at least a part of the posterior face of the lamina of said half of the vertebra, and which includes both a first peripheral portion, which is located substantially in anteroposterior line with the lower edge of the lamina of said one half of the vertebra when the coupling face is applied to said one half of the vertebra, and a second peripheral portion, which is located substantially in anteroposterior line with the lateral edge of the isthmus of said one half of the vertebra when the coupling face is applied to said one half of the vertebra. The coupling face further includes a first positioning stud, which protrudes from the first peripheral portion of the morphology-adapted surface, and a second positioning stud, which protrudes from the second peripheral portion of the morphology-adapted surface. The morphology-adapted surface and the first and second positioning studs are shaped so as to cooperate by matching shapes with the posterior surface of the lamina, the lower edge of the lamina and the lateral edge of the isthmus of said one half of the vertebra, respectively, so as to maintain the body in a unique position on the vertebra, when the coupling face is applied to said one half of the vertebra.

One of the ideas underlying the invention is to propose a patient-specific surgical guide, which can be used only on a hemivertebra, i.e. only on a half, either left or right, of a vertebra of the patient. Being thereby hemivertebral, the surgical guide according to the invention limits the surgical approach, by being usable in particular during a minimally invasive surgical procedure and/or during a unilateral intervention on the vertebrae. To this end, the body of the surgical guide according to the invention is designed for being coupled in a stable manner in a single fixed position, cooperating by matching contact with three anatomical areas of the relevant half of the vertebra, namely the posterior face of the lamina of said half of the vertebra, the lower edge of the lamina and the lateral edge of the isthmus of said half of the vertebra. Since the three anatomical areas have respective spatial orientations which are different from each other, the coupling of the body with the half of the vertebra is fixed in position, while the coupling can be achieved only according to a single predetermined positioning, due to the shaping of three corresponding coupling elements of the body of the surgical guide according to the invention, namely a surface, morphology-adapted to the posterior face of the lamina, a positioning stud to be positioned against the lower edge of the lamina, and a positioning stud to be positioned against the lateral edge of the isthmus. The shaping of the three aforementioned coupling elements is specific to the patient, being determined from preoperative mapping data relating to the concerned half of the vertebra, in particular to the three aforementioned anatomical areas of the latter. The distal face of the body of the surgical guide according to the invention, which carries the three aforementioned coupling elements, is squat, which allows the body to be particularly compact. In practice, the ease of use, as well as the compactness of the surgical guide according to the invention, are advantageously reinforced through arrangements of the body thereof, as discussed in detail below. In all cases, the body incorporates, in a proximal part, an aiming passage for guiding a surgical instrument along a planned trajectory planned preoperatively from preoperative mapping data relating to half of the vertebra: thereby, when the body of the surgical guide according to the invention is coupled to half of the vertebra, the aiming passage thereof provides guiding along said trajectory, in particular for centering the application of the surgical instrument on the pedicle of the half of the vertebra.

According to advantageous additional features of the surgical hemivertebral guide according to the invention, taken individually or according to all technically possible combinations:

The first positioning stud has a lateral surface which extends from the first peripheral portion of the morphology-adapted surface and which is shaped so as to extend tangentially to at least a portion of the lower edge of the lamina of said one half of the vertebra when the coupling face is applied to said one half of the vertebra, and the second positioning stud has a lateral surface which extends from the second peripheral portion of the morphology-adapted surface and which is shaped so as to extend tangentially to at least a portion of the lateral edge of the isthmus of said one half of the vertebra when the coupling face is applied to said one half of the vertebra.

The respective lateral surfaces of the first and second positioning studs extend from the morphology-adapted surface substantially parallel to a same geometric axis, the geometric axis extending in a substantially anteroposterior direction when the coupling face is applied to said one half of the vertebra.

The morphology-adapted surface includes, apart from the first and second peripheral portions thereof, a main region which is located in anteroposterior line with the posterior face of the lamina of said one half of the vertebra when the coupling face is applied to said one half of the vertebra, the main region being essentially convex, and the respective lateral surfaces of the first and second positioning studs being both turned towards and oriented transversely to the main region of the morphology-adapted surface.

The aiming passage is arranged with respect to the coupling face so as to be aligned with the pedicle of said one half of the vertebra when the coupling face is applied to said one half of the vertebra.

The body is further provided with at least one cannula suitable for receiving a removable bone anchor, such as a pin.

The at least one cannula extends lengthwise substantially parallel to the aiming passage.

The body includes a proximal part that delimits the aiming passage, a distal part that delimits the coupling face, and an intermediate part, which fixedly links the proximal and distal parts to each other and which is tapered.

The body is made by additive manufacturing.

### BRIEF DESCRIPTION OF THE DRAWINGS

The invention will be better understood upon reading the following description, given only as an example and making reference to the drawings, wherein:

The FIGS. 1 and 2 are posterior and upper views, respectively, of a vertebra of a patient;

FIG. 3 is a perspective view of a hemivertebral surgical guide according to the invention;

The FIGS. 4 and 5 are plan views according to the arrows IV and V respectively, as shown in FIG. 3;

FIG. 6 is a view similar to FIG. 1, showing the use of the guide shown in FIG. 3, on the vertebra shown in FIG. 1;

The FIGS. 7 and 8 are schematic partial sections along the lines VII-VII and VIII-VIII, respectively, as shown in FIG. 6; and

FIG. 9 is a view similar to FIG. 6, from a different angle of observation.

### DESCRIPTION

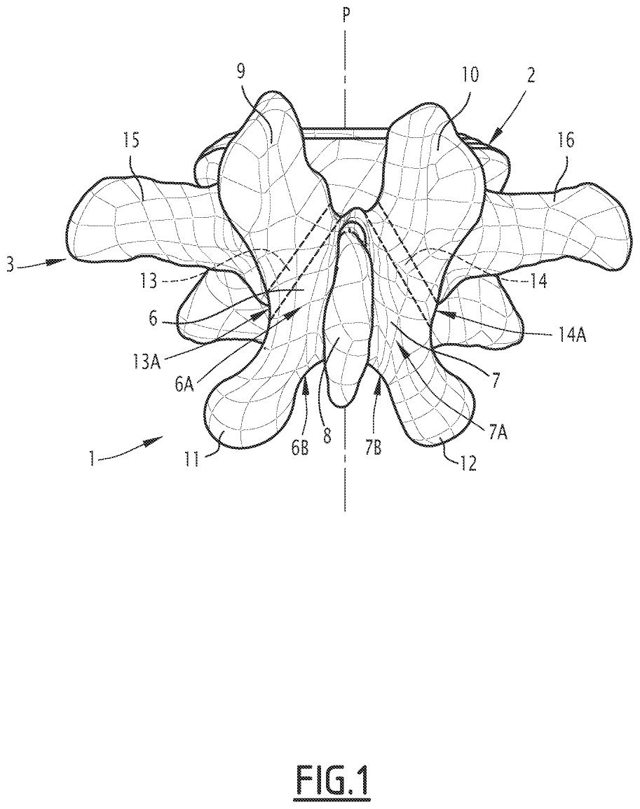

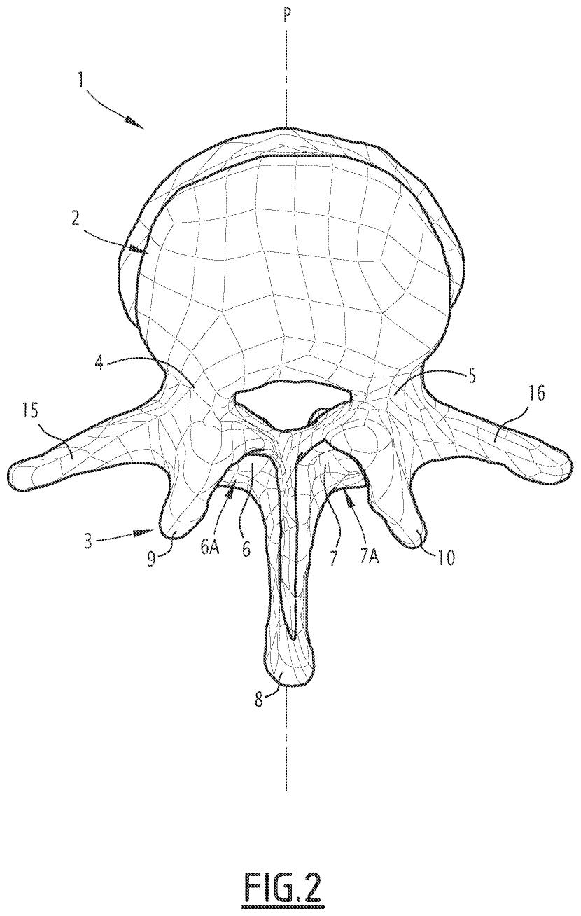

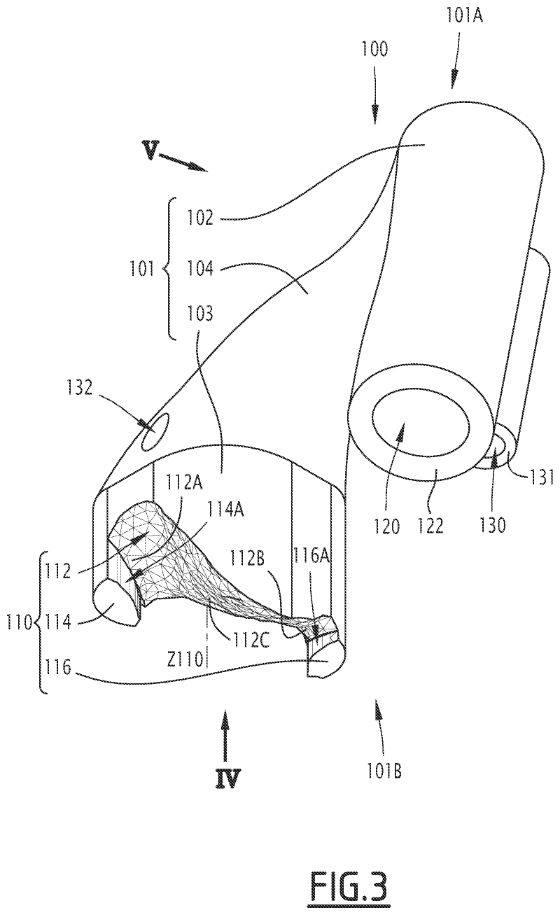

Before describing in detail an example of a surgical guide according to the invention, with reference to FIGS. 3 to 9, certain anatomical aspects of human vertebrae are recalled below with reference to FIGS. 1 and 2 wherein a vertebra 1 of a human patient is schematically represented.

The vertebra 1 includes, at the front, a vertebral body 2 and, at the back, a vertebral arch 3. The vertebral arch 3 includes, on both sides of the sagittal plane P of the vertebra 1, a left pedicle 4 and a right pedicle 5, which join the vertebral body 2 to the rest of the vertebral arch 3, each extending overall along an anteroposterior direction. The vertebral arch 3 further includes, on both sides of the sagittal plane P, a left lamina and a right lamina 7, which extend both rearwards and inwards, respectively, from the left pedicle 4 and the right pedicle 5, until reaching, at the sagittal plane P, a massive spinous process 8, belonging to the vertebral arch 3. On the posterior side of the vertebral arch 3, the left lamina 6 has a posterior face 6A and the right lamina 7 has a posterior face 7A.

The vertebral arch 3 further includes a left upper articular process 9 and a right upper articular process 10, each extending upwards from the left lamina 6 and the right lamina 7, respectively, as well as a left lower articular process 11 and a right lower articular process 12, which each extends downwards from the left lamina 6 and the right lamina 7, respectively. The left 9 and right 10 upper articular processes are articulated with the left and right lower articular processes of a neighboring vertebra (not shown), stacked above the vertebra 1 within the patient's spine, while the left 11 and the right 12 lower articular processes are articulated with the left and right upper articular processes of another nearby vertebra (not shown), stacked below the vertebra 1 within the spine. On the lower side of the vertebral arch 3, the left lamina 6, and the right lamina 7, respectively, has a lower edge 6B, and 7B, respectively, which links the spinous process 8 to the left lower articular process 11, and to the right lower articular process 12, respectively.

Moreover, as shown with dotted lines in FIG. 1, the vertebral arch 3 forms, at the constricted junction between the left lamina 6 and the left upper articular process 9, a left isthmus 13 and forms, at the constricted junction between the right lamina and the right upper articular process 10, a right isthmus 14. On the left, and right side respectively of the vertebra 1, the left 13, and the right 14 isthmus, respectively, have a lateral edge 13A, and 14A, respectively, which corresponds substantially to the part of the left 13, and right 14 isthmus, respectively, which is furthest from the spinous process 8.

Finally, the vertebral arch 3 further includes a left transverse process 15 that extends to the back and outwards from the left pedicle 4, and a right transverse process 16 that extends outwards and to the back from the right pedicle 5.

The vertebra 1 consists of two hemivertebrae, left and right, respectively, which correspond to the two halves of the vertebra 1, located on both sides of the sagittal plane P. Thereby, the left hemivertebra of the vertebra 1 comprises the left half of the vertebral body 2 and the left half of the spinous process 8, as well as the left pedicle 4, the left lamina 6, the left upper articular process 9, the left lower articular process, the left isthmus 13 and the left transverse process 15.

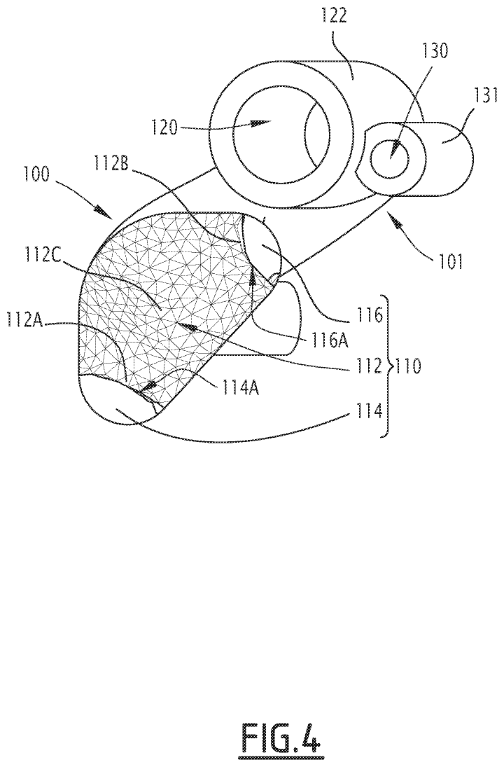

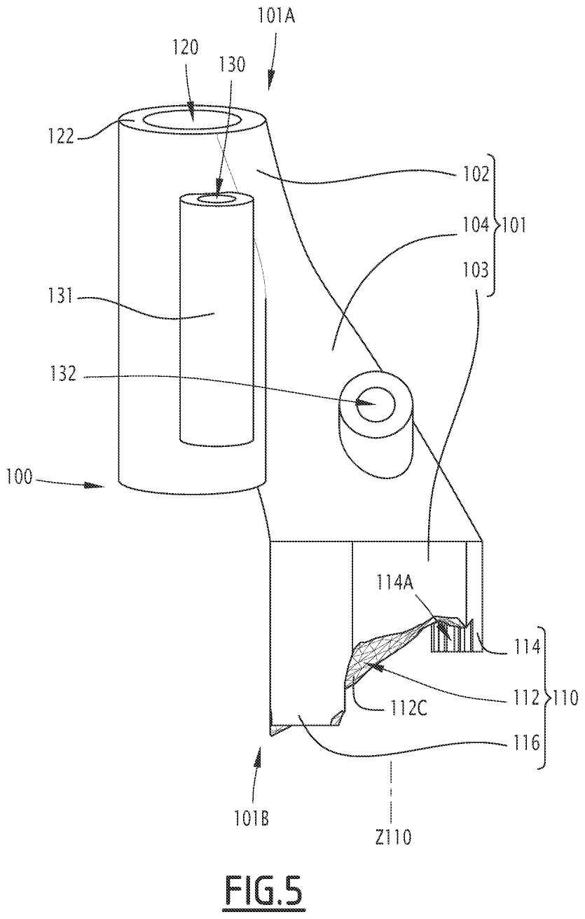

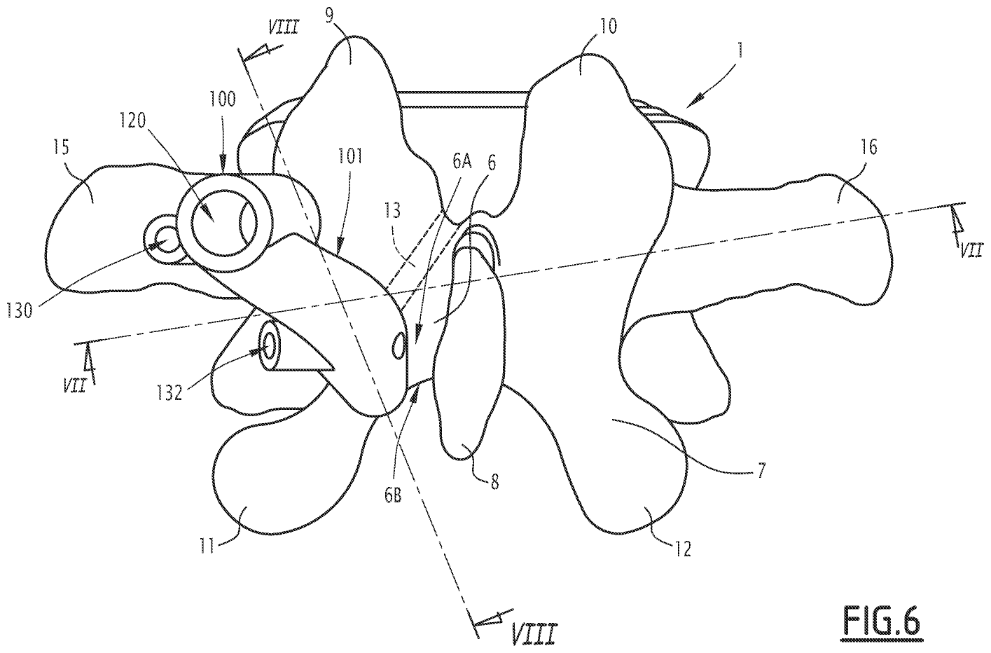

FIGS. 3 to 9 show a surgical guide 100. The surgical guide 100 is hemivertebral, in the sense that, as explained in detail below, the guide is designed for being applied exclusively to one half, either left or right, of a vertebra, without interacting with the other half. Moreover, the surgical guide 100 is patient-specific, in the sense that, also as explained below, the guide is designed in a personalized manner for a given patient, by being bespoke for corresponding to the negative bone topography of certain areas of a given vertebra of the patient concerned, more precisely of a given hemivertebra of the patient. As an example on which the rest of the description is based, the surgical guide 100 is thus specifically adapted to the left hemivertebra of the vertebra 1 of FIGS. 1 and 2, the vertebra 1 being shown schematically in FIGS. 6 to 9 where the surgical guide 100 is applied to the left hemivertebra of said vertebra.

The surgical guide 100 includes a body 101 that has opposite sides, a proximal 101A and distal 101 B side, respectively. When the surgical guide 100 is used on the vertebra 1, the distal side 101B is turned towards the posterior side of the vertebra, more precisely the left hemivertebra of the latter.

In the embodiment considered in the figures, the body 101 includes a proximal part 102 and a distal part 103, which are fixedly linked to each other by an intermediate part 104 of the body 101, which is advantageously tapered.

The body 101 is provided, on the distal side 101B thereof, with a coupling face 110 that is herein delimited by the distal part 103. The coupling face 110 makes it possible to mechanically couple the body 101 to the bone material of the left hemivertebra of the vertebra 1 and is specific to the patient so that same can be applied in bespoke contact with the bone material. To this end, the coupling face 110 includes a morphology-adapted surface 112 and two positioning studs 114 and 116.

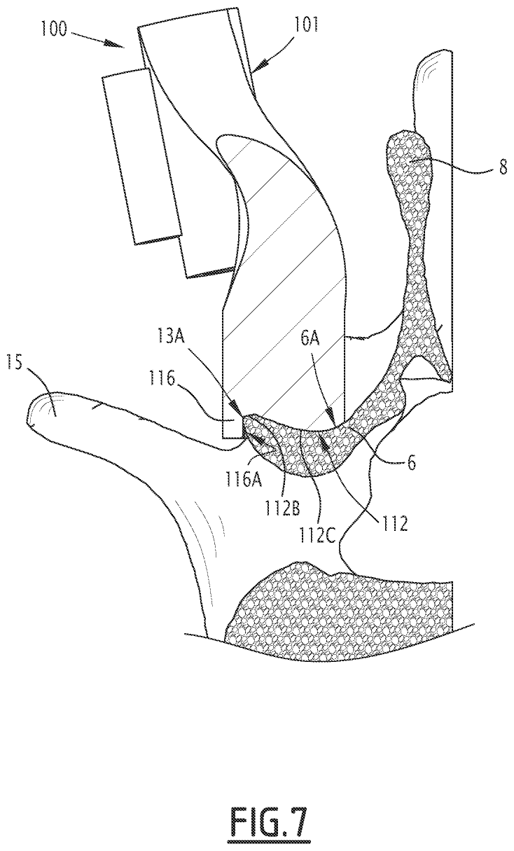

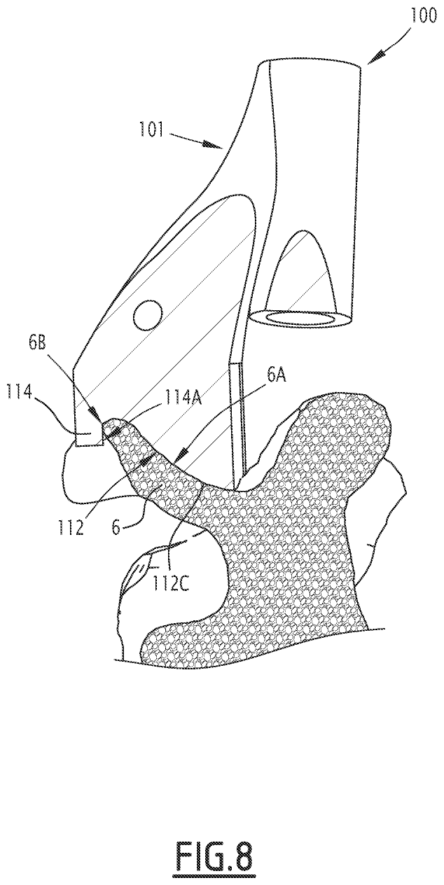

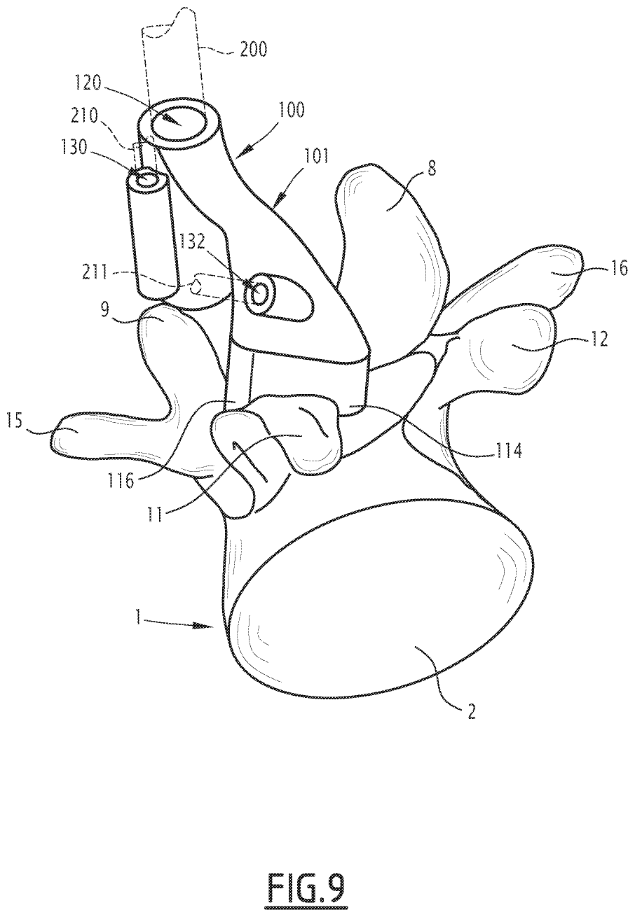

The morphology-adapted surface 112 is congruent with at least a portion of the posterior face 6A of the left lamina 6. When the coupling face 110 is applied to the left hemivertebra of the vertebra 1, the morphology-adapted surface 112 covers at least part of the posterior face 6A of the left lamina 6, fitting the bony reliefs thereof. As can be seen clearly in FIGS. 3 to 5, the morphology-adapted surface 112 has an extent such that same includes two peripheral portions 112A and 112B, which are distinct from each other, being distributed along the periphery of the morphology-adapted surface 112: when the coupling face 110 is applied to the left hemivertebra of the vertebra 1, the peripheral portion 112A is located substantially in front of the lower edge 6B of the left lamina 6, as can be seen clearly in FIGS. 8 and 9, whereas the peripheral portion 112B is located substantially in front of the anteroposterior vertical line with the lateral edge 13A of the left isthmus 13, as can be seen clearly in FIGS. 7 and 9. Outside the peripheral portions 112A and 112B, the morphology-adapted surface 112 includes a main region 112C which, when the coupling face 110 is applied to the left hemivertebra of vertebra 1, is located in anteroposterior line with the posterior face 6A of the left lamina 6: the main region 112C is designed fitted to the central zone of the posterior face 6A of the left lamina. To this end, the main region 112C is advantageously essentially convex.

As can be seen clearly in FIGS. 3 to 5, the positioning stud 114 protrudes from the peripheral portion 112A of the morphology-adapted surface 112 and the positioning stud 116 protrudes from the peripheral portion 112B of the morphology-adapted surface. In the advantageous embodiment illustrated in the figures, the positioning studs 114 and 116 include a lateral surface 114A, 116A respectively, which extends from the peripheral portion 112A, and from the peripheral portion 112B, respectively. The lateral surfaces 114A and 116A are shaped so as to extend, with regard to the lateral surface 114A, tangentially to at least a part of the lower edge 6B of the left lamina 6, as can be seen clearly in FIG. 8, and, with regard to the lateral surface 116A, tangentially to at least part of the lateral edge 13A of the left isthmus 13, as clearly visible in FIG. 7, when the coupling face 110 is applied to the left hemivertebra of the vertebra 1. According to a practical and effective possibility, the lateral surfaces 114A and 116A of the positioning studs 114 and 116 extend from the morphology-adapted surface 112 substantially parallel to the same geometric axis Z110 which, when the coupling face 110 is applied to the left hemivertebra of the vertebra 1, extends along a substantially anteroposterior direction. The lateral surfaces 114A and 116A of the positioning studs 114 and 116 are both turned towards and oriented transversely with regard to the main region 112C of the morphology-adapted surface 112, i.e. not parallel, to the main region 112C, as can be seen clearly in FIG. 3.

In all cases, when the coupling face 110 is applied to the left hemivertebra of the vertebra 1, the morphology-adapted surface 112 and the positioning studs 114 and 116 are shaped so as to cooperate by matching shapes with the posterior face 6A of the left lamina 6, the lower edge 6B of the left lamina 6 and the lateral edge 13A of the left isthmus 13, respectively, so as to maintain the body 101 in a unique position on the vertebra 1. Indeed, the uniqueness of the positioning results from the adjustment of shapes between the posterior face 6A of the left lamina and the morphology-adapted surface 112, in particular the main region 112C of the latter. Moreover, in an overall medial-lateral direction, the coupling face 110 is held fixedly in position on the left hemivertebra, by means, in one direction, of the abutment of the lateral edge 13A of the left isthmus 13 against the positioning stud 116, in particular against the lateral face 116A of the latter, and by means, in the opposite direction, of the abutment of the posterior face 6A of the left lamina against the morphology-adapted surface 112, in particular the main region 112C of the latter, as illustrated in FIG. 7. In an overall vertical direction, the coupling face 110 is held fixedly in position on the left vertebrae, by means, in one direction, of the abutment of the lower edge 6B of the left lamina against the positioning stud 114, in particular the lateral surface 114A of the latter, and by means, in the opposite direction, of the abutment of the posterior face 6A of the left lamina 6 against the morphology-adapted surface 112, in particular the main region 112C of the latter, as illustrated in FIG. 8. Finally, in an overall anteroposterior direction, the coupling face 110 is held fixedly in position on the left hemivertebra of the vertebra 1 towards the front by means of the abutment of the posterior face 6A of the left lamina 6 against the morphology-adapted surface 112, whereas, backwardly, the body 101 remains removable with regard to the vertebra.

In practice, the morphology-adapted surface 112 and the positioning studs 114 and 116 are shaped preoperatively, using preoperative mapping data relating to the vertebra 1, in particular to the left lamina 6 and the left isthmus 13 thereof. Such preoperative mapping data are made available by ad hoc mapping means, coming e.g. from one or a plurality of scanner images belonging to the mapping means, i.e. from one or a plurality of tomographic images obtained by preoperative irradiation of the vertebra 1. In all cases, the preoperative mapping data form a morphological meshing of the bone structure of the vertebra 1. Since the acquisition of such preoperative mapping data is well known in the field, same will not be discussed in further detail herein. Once the preoperative mapping data is acquired, same is used preoperatively for shaping the morphology-adapted surface 112 and the positioning studs 114 and 116 so that the morphology-adapted surface 112 and the positioning studs 114 and 116 adopt the topographic negative of the morphological meshing of the left lamina 6 and the left isthmus 13, as discussed in detailed hereinabove. The shaping is carried out during the production of the coupling face 110 by any appropriate manufacturing technique, in particular by additive manufacturing otherwise called three-dimensional printing, in a manner known per se.

The body 101 of the surgical guide 100 is, moreover, provided with an aiming passage 120 that is delimited herein by the proximal part 102 of the body 101. The aiming passage 120 links the proximal side 101A and the distal side 101 B of the body 101. The specificities of the aiming passage 120 are not limiting provided same is suitable for guiding a surgical instrument, such as a bone drilling tool or a bone implant tool. In the example of embodiment considered in the figures, the aiming passage 120 is formed by the internal volume, herein cylindrical, of a tubular element 122 integrated into the body 101.

In practice, the arrangement of the aiming passage 120 with respect to the coupling face 110 is predetermined so that the aiming passage 120 guides the aforementioned surgical instrument along a precise trajectory with respect to the vertebra 1. In particular, according to a preferred embodiment that is shown in the figures, the aiming passage 120 is arranged with respect to the coupling face 110 so as to be aligned with the left pedicle 4 when the coupling face 110 is applied to the left hemivertebra of the vertebra 1. To this end, the arrangement of the aiming passage 120 is planned using the aforementioned preoperative mapping data. Such planning is made possible by ad hoc planning means, such as a software device, which calculates planning data, which are defined with respect to the preoperative mapping data and which define the trajectory of the surgical instrument guided by the aiming passage 120. The trajectory is e.g. characterized by the orientation, with respect to the vertebra 1, of the aiming passage 120 and, thereby, of the surgical instrument, and the projection of the aiming passage 120 according to said orientation on the vertebra 1, in other words the point of entry of the surgical instrument into the vertebra 1. In all cases, the planning data are used when obtaining the aiming passage 120 by any appropriate manufacturing technique, in order to arrange the aiming passage 120 in an appropriate manner with respect to the coupling face 110.

In the embodiment considered in the figures, the body 101 of the surgical guide 100 is further provided with one or a plurality of cannulas, which are herein in number of two and which are referenced by 130 and 132, respectively. The cannula 130 extends lengthwise substantially parallel to the aiming passage 120 and is delimited herein by the proximal part 102 of the body 101, being formed by the cylindrical internal volume of a tubular element 131 carried externally by the tubular element 122. The cannula 132 is delimited by the intermediate part 104 of the body 101, extending lengthwise transversely, i.e. not parallel, to the cannula 130. Each of the cannulas 130 and 132 receives a removable bone anchoring element, such as a pin. Thus, when using the surgical guide 100 on the vertebra 1 and once the coupling face 110 is applied to the left hemivertebra of the vertebra 1, the body 101 can, if necessary, be locked onto the vertebra 1 by inserting a pin into the vertebra 1 via the cannula 130 and/or by inserting another pin into the vertebra 1 via the cannula 132. In such way, the body 101 does not have to be held manually during the application of the surgical instrument to the vertebra 1 via the aiming passage 120, and there is no risk that the surgical guide 100 moves with respect to the vertebra. The body 101 can thus advantageously be devoid of corresponding gripping elements, such as a handle.

An example of implementation of the surgical guide 100 will now be described.

During a preoperative phase, the body 101 of the surgical guide 100 is manufactured from the preoperative mapping data relating to the vertebra 1, as mentioned hereinabove. As explained in detail hereinabove, the embodiment of the body 101, which is preferentially implemented by additive manufacturing, in particular by three-dimensional printing, results in having the coupling face 110, making same patient-specific, more precisely specific to the left hemivertebra of the vertebra 1 of the patient, and having the aiming passage 120, making same apt for guiding a surgical instrument along a planned trajectory with respect to the vertebra 1.

During an intraoperative phase, the patient to be operated is anesthetized, e.g. while lying on his/her stomach on an operating table. The surgeon then performs the approach of the vertebra 1 so as to expose, without altering same, an area of the vertebra 1. The exposed area is thus freed from any soft part, such as skin, flesh, etc., so as to leave the bone material of the area of the vertebra 1 exposed to the air. The area thus exposed includes at least three anatomical regions of the left hemivertebra of the vertebra 1, namely the posterior face 6A of the left lamina 6, the lower edge 6B of the left lamina 6 and the lateral edge 13A of the left isthmus 13. The exposed zone can advantageously be limited to the only three anatomical regions thereof, thus being exposed by an approach route which can be described as minimally invasive and which concerns only one side, herein the left, of the patient's spine, with a reduced lateralized incision. As a variant, the exposed zone can, of course, be more extensive, being then exposed by a wide-open approach route, involving, where appropriate, the two lateral sides of the spine. In all cases, it should be noted that the exposure of the three anatomical regions mentioned above is facilitated by the fact that the three regions are not the place of insertion of any ligament, such as the ligamentum flavum.

The surgeon then uses the surgical guide 100 on the vertebra 1, by applying the coupling face 110 on the exposed zone so as to place the body 101 on the vertebra 1 in the unique position defined hereinabove. The surgical system 100 is then in the configuration illustrated in FIGS. 6 to 9. As explained in detail above, the coupling of the body 101 to the left hemivertebra of the vertebra 1 is fixedly stabilized in such unique position because of the cooperation of shapes of the three aforementioned anatomical regions with the morphology-adapted surface 112 and the positioning studs 114 and 116. Once coupled to the left hemivertebra of the vertebra 1, the surgical guide 100 can be released by the surgeon, while remaining fixedly in place on the vertebra 1. The surgeon can then easily visually inspect the surgical site, especially since the body 101 is particularly compact, in particular due to the tapering of the intermediate part 104 thereof.

The surgeon then uses a surgical instrument, such as a bone drilling tool or a bone implant tool, which the surgeon applies to the vertebra 1 by guiding same through the aiming passage 120. Only in FIG. 9, such surgical instrument is shown schematically, being referenced by 200. If necessary, before using the surgical instrument 200, the surgeon locks in position, the body 101 of the surgical guide 100 on the vertebra 1 by means of one or more removable bone anchors inserted into the vertebra via one and/or the other of the cannulas 130 and 132: only in FIG. 9, two such removable bone anchoring elements are shown schematically, being referenced by 210 and 211, respectively.

After applying the surgical instrument 200, the surgeon removes the surgical guide 100 from the vertebra 1, where appropriate by first removing the removable bone anchoring elements 210 and 211.

Moreover, various arrangements and variants or options concerning the surgical guide 100, and the use thereof, described so far, can be envisaged. Examples include:

as mentioned above, the surgical guide 100 can have a version for a right hemivertebra; and/or

other types of manufacturing than additive manufacturing can be used for manufacturing all or part of the surgical guide 100.

1. A patient-specific hemivertebral surgical guide, including a body which is provided with:
an aiming passage, which links proximal and distal sides of the body to each other and which is suitable for guiding a surgical instrument, and
a coupling face, which is arranged on the distal side of the body and which is patient-specific to be applied in bespoke contact with bone material of only one half, either left or right, of a vertebra of the patient,
wherein the coupling face includes:
a morphology-adapted surface, which is congruent with at least a part of the posterior face of the lamina of said one half of the vertebra and which includes both a first peripheral portion, which is located substantially in anteroposterior line with the lower edge of the lamina of said one half of the vertebra when the coupling face is applied to said one half of the vertebra, and a second peripheral portion, which is located substantially in anteroposterior line with the lateral edge of the isthmus of said one half of the vertebra when the coupling face is applied to said one half of the vertebra,
a first positioning stud protruding from the first peripheral portion of the morphology-adapted surface, and
a second positioning stud protruding from the second peripheral portion of the morphology-adapted surface,
and wherein the morphology-adapted surface and the first and second positioning studs are shaped so as, when the coupling face is applied to said one half of the vertebra, to cooperate by matching shapes with the posterior face of the lamina, the lower edge of the lamina and the lateral edge of the isthmus of said one half of the vertebra, respectively, so as to maintain the body in a unique position on the vertebra.

2. The surgical guide according to claim 1,
wherein the first positioning stud has a lateral surface, which extends from the first peripheral portion of the morphology-adapted surface and which is shaped so as to extend tangentially to at least a portion of the lower edge of the lamina of said one half of the vertebra when the coupling face is applied to said one half of the vertebra,
and wherein the second positioning stud has a lateral surface which extends from the second peripheral portion of the morphology-adapted surface and which is shaped so as to extend tangentially to at least a part of the lateral edge of the isthmus of said one half of the vertebra when the coupling face is applied to said one half of the vertebra.

3. The surgical guide according to claim 2, wherein the respective lateral surfaces of the first and second positioning studs extend from the morphology-adapted surface substantially parallel to a same geometric axis, the geometric axis extending in a substantially anteroposterior direction when the coupling face is applied to said one half of the vertebra.

4. The surgical guide according to claim 2,
wherein the morphology-adapted surface includes, apart from the first and second peripheral portions thereof, a main region which is located in anteroposterior line with the posterior face of the lamina of said one half of the vertebra when the coupling face is applied to said one half of the vertebra, the main region being substantially convex,
and wherein the respective lateral surfaces of the first and second positioning studs are both turned towards and oriented transversely to the main region of the morphology-adapted surface.

5. The surgical guide according to claim 1, wherein the aiming passage is arranged with respect to the coupling face so as to be aligned with the pedicle of said one half of the vertebra when the coupling face is applied to said one half of the vertebra.

6. The surgical guide according to claim 1, wherein the body is further provided with at least one cannula suitable for receiving a removable bone anchor.

7. The surgical guide according to claim 6, wherein the at least one cannula extends longitudinally substantially parallel to the aiming passage.

8. The surgical guide according to claim 1, wherein the body includes:
a proximal part which delimits the aiming passage,
a distal part which delimits the coupling face, and
an intermediate part, which fixedly links the proximal and distal parts to each other, and which is tapered.

9. The surgical guide according to claim 1, wherein the body is made by additive manufacturing.
